(19) United States
(12) Patent Application Publication
(10) Pub. No.: US 2008/0115260 A1
(43) Pub. Date: May 22, 2008

(54) INTEGRAL HELMET

(76) Inventors: Christian SCHULZ, Liebenburg (DE); Oliver SCHIMPF, Braunschweig (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/772,639

(22) Filed: Jul. 2, 2007

(30) Foreign Application Priority Data
Jul. 4, 2006 (DE) 10 2006 031 751.3

(51) Int. Cl. A42B 7/00; A42B 1/08; A42B 3/00; A61F 9/02

(52) U.S. Cl. 2/421; 2/424; 2/425; 2/427

(57) ABSTRACT

In the case of an integral helmet with a helmet shell (1) which protects the head, with a visor opening (2) and with a chin part (4) which defines the lower side of the visor opening (2) and is provided with a plurality of functional elements and at least partially with a decorative surface, simpler handling and savings on weight can be achieved in that the chin part (4) is formed with a supporting part (8) which carries the functional elements, and in that the decorative surface is formed by at least one surface part (9) which is connected to the supporting part (8).

The invention relates to an integral helmet with a helmet shell which protects the head, with a visor opening and with a chin part which defines the lower side of the visor opening and is provided with a plurality of functional elements and at least partially with a decorative surface.

Integral helmets are distinguished in that they cover the helmet wearer's chin region below the visor opening and therefore protect the chin region against impacts, knocks or the like, for example should a motorcyclist take a fall.

Integral helmets are known, in which the chin part is realized integrally with the helmet shell. Accordingly, the chin part, like the helmet shell, is composed of a stable plastic which can be produced by injection molding or by formation of a laminate, for example using thermosetting plastics.

Furthermore, integral helmets are known, in which the chin part is movable relative to the helmet shell in order, in the closed state of the chin part, to permit a close fit of the helmet inner lining around the head and in the neck region of the helmet wearer, but also to permit easy removal of the integral helmet from the helmet wearer's head after an accident by the chin part being opened by a movement away from the helmet shell. In particular, chin parts which can be swung up have been successful for this purpose.

Even in the case of integral helmets with chin parts which are movable relative to the helmet shell, the chin part is usually produced in the same manner as the helmet shell, i.e. from a stable material. This material has—at least in part—to have a considerable material thickness in order to be able to fasten fastening means and other functional elements to the chin part. A movable chin part has to be connectable in a stable manner to the helmet shell in order, in the case of an accident, to retain the closed shape of the integral helmet and thus to ensure the sought protection during the accident. Furthermore, it has proven worthwhile to introduce fresh air into the interior of the helmet via the chin part, with it being necessary for the supply of fresh air to be able to be switched on and switched off in order to take account of different external temperatures and ventilation requirements of the helmet wearer. It is therefore known to provide a fresh air opening on the chin part, which fresh air opening is covered by a closure element and through which fresh air is conducted along the inside of the swung-down visor panel into the interior of the integral helmet. By actuating an adjustment element, the supply of fresh air can be prevented or adjusted in terms of quantity of fresh air.

It is furthermore known, in order to remove the integral helmet, to open the locking of the chin part to the helmet shell—customarily at both ends of the chin part that butt against the helmet shell—by means of a common release button, the release button actuating the two locking elements by means of a Bowden cable, for example. The Bowden cable or a sheath guiding the Bowden cable has to be guided on the inside of the chin part from the release button to the locking elements. The guides, as functional elements, are therefore realized in the chin part, for example by adhesive bonding. The chin part—like the helmet shell—is generally painted on the outside in order to provide the integral helmet with a decorative surface in the region of the helmet shell and in the region of the chin part. Since a plurality of functional elements, in particular also the closure devices, have to be fastened to the chin part—generally after the surface has already been painted, the fitting of the functional elements and the handling of the chin part prove complicated, since the paint must not be damaged. Furthermore, the chin part material, which firstly has the required stability and secondly has to be designed in order to receive fastening elements, has to be of relatively solid design, as a result of which the chin part contributes not inconsiderably to the overall weight of the integral helmet.

The present invention is based on the object of designing an integral helmet of the type mentioned at the beginning in such a manner that the chin part can be produced with a saving on weight and with simplified handling.

To achieve this object, an integral helmet of the type mentioned at the beginning is characterized, according to the invention, in that the chin part is formed with a supporting part which carries the functional elements, and in that the decorative surface is formed by at least one surface part which is connected to the supporting part.

The present invention therefore provides a separation of the functions of the chin part by a supporting part serving for the fastening of the functional elements and for ensuring the required stability of the chin part while the decorative surface is formed by the at least one surface part which is connected to the supporting part by a suitable joining technique—for example latching or adhesive bonding—and which does not necessarily have to have a particular stability. The connection may also be designed in such a manner that a later exchange of the surface part or of the surface parts is possible.

The supporting part may be designed as an injection-molded part and therefore everything can be provided during the shaping operation for the functional elements. The supporting part can be constructed in a particularly stable manner by suitable shapings, such as ribbed structures, etc., with a saving on weight being possible in that the supporting part is provided in the manner of a framework with recesses which do not impair the stability of the supporting part, but reduce the weight and the use of material. The realization of the decorative surface by a corresponding surface part or a plurality of surface parts of this type, which are connected to the supporting part, for example by means of latching elements, makes it possible for the surface parts to be separately painted and for the surface parts to be fitted to the supporting part only after all of the other manipulations required at the chin part have been carried out, and therefore there is no risk of the decorative surface of the surface parts being damaged by the chin part handling that is still required during the production process of the integral helmet.

The supporting part may, in particular, already be formed with fastening elements which form parts of a pivot joint for swinging up the chin part. In a corresponding manner, locking devices for the fixed connection of the chin part to the helmet shell are fastened to the supporting part in a suitable manner.

In a further preferred embodiment, the supporting part has a ventilation opening and a mounting for a ventilation element which controls the ventilation.

An exemplary embodiment of the invention will be explained in more detail below with reference to the attached drawing, in which:

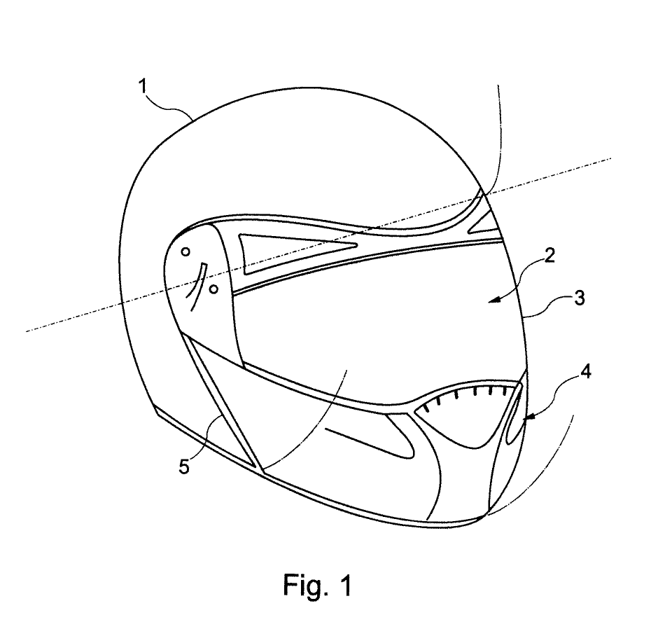

FIG. 1 shows an integral helmet with a chin part which can be swung up, in the closed position,

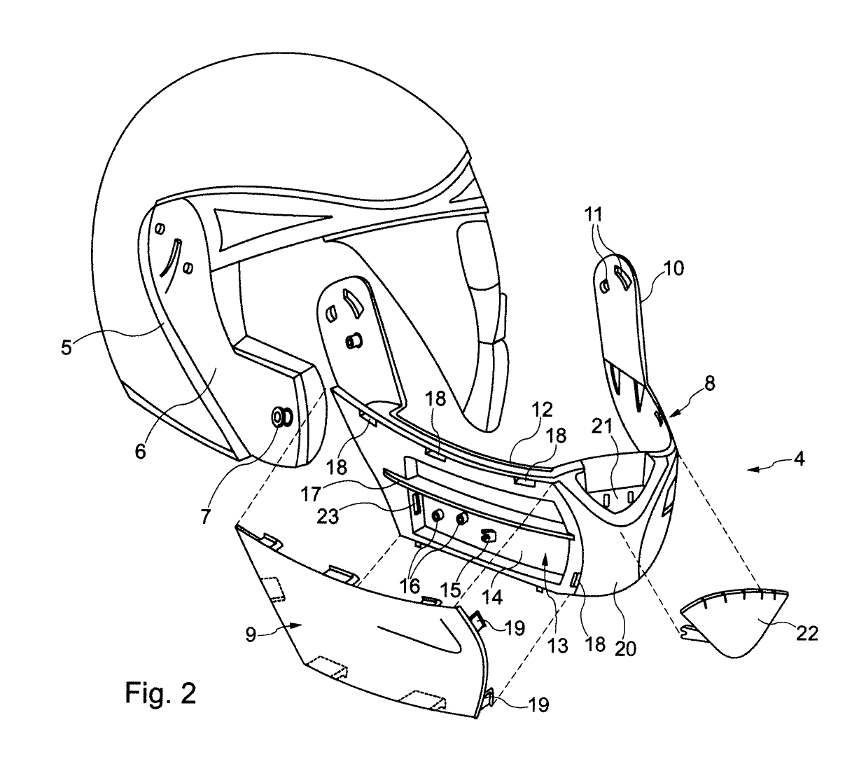

FIG. 2 shows the integral helmet from FIG. 1 in an exploded illustration of the parts of the chin part.

The complete integral helmet illustrated in FIG. 1 has a helmet shell 1 which customarily extends from the front region over the upper part of the helmet wearer's head and down into the region at the back of his neck. The helmet shell forms a visor cut-out 2 which is closed by a transparent visor panel 3. The visor panel 3 is generally designed such that it can be swung up independently.

The lower edge of the visor opening 2 is defined by a chin part 4 which bears with rearwardly pointing ends against abutting edges 5 of the helmet shell 1 and can be locked there by locking elements (not illustrated) in the closed form illustrated in FIG. 1. For this purpose, the helmet shell 1 has, on an inwardly offset bearing surface 6 a mushroom-shaped bolt 7 which can be engaged with a snap fit by a locking mechanism arranged on the chin part 4. FIG. 2 clarifies the individual parts of the chin part 4 without built-in functional elements. According thereto, the chin part 4 is composed of a stable supporting part 8 and two surface parts 9, only one of which is illustrated in FIG. 2 for the sake of clarity.

The supporting part 8 is of integral design and constitutes the element which determines the stability of the chin part 4. Accordingly, the supporting part 8 has the shape of the chin part and, at its rear ends, comprises two inwardly offset planar extension pieces 10 on which are located parts 11 of a pivot joint with which the chin part 4 can be fastened to the helmet shell 1 in a manner such that it can be swung up out of the position of FIG. 1. Below the extension pieces 10 there is a basic body 12 of the supporting part 8, which, in its basic form, is designed symmetrically with respect to a central plane of the integral helmet 1 and, on its two limbs which are directed in a manner such that they are bent rearwards, has a type of framework structure by a recess 13, by means of which a frame-shaped structure is formed, being provided on the two limbs. The recess 13 may be formed by a step-like reduction in the material thickness but also by a wall-type guide, which is pivoted out of the plane, with the material thickness remaining approximately the same. The recess 13 is therefore open towards the outside and has a rear wall 14 which is connected, preferably integrally, to retaining devices 15, 16. Furthermore, a supporting rib 17 which spans the recess 13 can be seen over the length of the recess 13.

The supporting part 8 has an arrangement of latching slots 18 which interact with latching hooks 19, fitted on the rear side of the surface part 9, in order to fasten the surface part 9 to the supporting part 8.

In the exemplary embodiment illustrated, the surface parts 9 do not completely cover the supporting part on its visible surface. On the contrary, in the center of the chin part 4, the supporting part is designed with a surface section 20 in such a manner that the surface section 20 is aligned with the surface parts 9, thus resulting in a two-colored design of the surface of the chin part 4, namely by means of the painted surface parts 9, on the one hand, and the surface section 20 which is generally formed without paintwork and therefore is generally in the color of the material of the retaining part 8, i.e., for example, is black.

The surface parts 9 can be produced to be very thin and light. They do not therefore contribute to the stability of the chin part 4. The risk of the surface part 9 buckling into the recess 13 is eliminated by the supporting rib 17 which bears in a supporting manner against the inner surface of the surface part 9.

The central surface section 20 is adjoined towards the visor opening 2 by a fresh air inlet 21 which can be covered by a switch rocker 22 as ventilation element. Since the switch rocker 22 is designed in a manner such that it can be latched into a plurality of positions, the supply of fresh air can take place with it in a metered manner and at a plurality of strengths or can be entirely switched off. The ventilation element 22 is therefore likewise mounted on the retaining part 8. This also applies to a release button (not illustrated) which can be arranged, for example, on the lower edge of the chin part 4 below the surface section 20 and actuates a Bowden cable which is guided in guide elements 15 within the recesses 13 to the locking elements at the rear ends of the chin part 4, and therefore the two locking elements can be released simultaneously by the centrally arranged release button in order to be able to swing up the chin part 4 in order to remove the integral helmet. In order to guide the Bowden cable, a lead-through opening 23 can be seen on a narrow side wall of the recess 13 in FIG. 1.

In the embodiment illustrated, the retaining part 8 contains all of the functional elements which are required for the chin part 4, in particular the formation of the pivot joint parts 11 on the extension pieces, the fastening of locking elements for locking the chin part 4 in the swung-down state, a release button and the release mechanism of the locking elements in the form of a Bowden cable, adjustable ventilation openings and retaining elements for an internal padding of the chin part 4. The surface parts 9 serve merely to cover the functional elements 15, 16, which can now be fitted to the retaining part 8 without taking into consideration a visual external effect, and afford the advantage that they can be painted separately, and therefore they are not subject to any risk of damage during the handling and installation of the supporting part 8.

The surface parts 9, as an injection-molded part, are preferably composed of a polycarbonate ABS blend as used, for example, for decoration shells for mobile telephones. The supporting part 8 is preferably an injection-molded part which can be formed from any suitable, stable material. Owing to the shaping and owing to ribbed structures, it can be produced with high torsional rigidity and with all of the fastening options which are required.

In the exemplary embodiment illustrated, two surface parts 9 are provided which, together with the surface section 20 of the supporting part 8, form the surface of the chin part 4. Of course, it is possible for one surface part 9 to be provided which covers the entire surface of the retaining part 8 if it is visible on the helmet. Furthermore, it is likewise possible to design the retaining part 8 in such a manner that it has a plurality of decorative surface sections 20, and therefore a correspondingly higher number of surface parts 9 is also provided.

1. Integral helmet with a helmet shell (1) which protects the head, with a visor opening (2) and with a chin part (4) which defines the lower side of the visor opening (2) and is provided with a plurality of functional elements and at least partially with a decorative surface, characterized in that the chin part (4) is formed with a supporting part (8) which carries the functional elements, and in that the decorative surface is formed by at least one surface part (9) which is connected to the supporting part (8).

2. Integral helmet according to claim 1, characterized in that the surface of the surface part (9) is painted.

3. Integral helmet according to claim 1, characterized in that the supporting part (8) is at least partially designed in the shape of a frame by means of recesses (13).

4. Integral helmet according to claim 1, characterized in that the supporting part is an injection-molded plastic part.

5. Integral helmet according to claim 1, characterized in that the supporting part (8) is provided with fastening elements (11) for fastening the chin part (4) to the helmet shell (1).

6. Integral helmet according to claim 4, characterized in that the fastening elements (11) form parts of a pivot joint for swinging up the chin part (4).

7. Integral helmet according to claim 1, characterized in that the supporting part (8) is provided with a mounting for a ventilation element (22).

8. Integral helmet according to claim 1, characterized in that locking elements for locking the chin part (4) to the helmet shell (1) in the closed state are mounted on the supporting part (8).
